(19) United States
(12) Patent Application Publication
(10) Pub. No.: US 2016/0089536 A1
(43) Pub. Date: Mar. 31, 2016

(54) Targeted And Individualized Delivery Of Skincare Treatments With Micro-Current In A Mask Or Patch Form

(71) Applicant: ELC Management LLC, Melville, NY (US)

(72) Inventors: Fatemeh Mohammadi, Hauppauge, NY (US); Tsung-wei Robert Mou, Stony Brook, NY (US); Lisa Qu, Flushing, NY (US)

(21) Appl. No.: 14/500,824

(22) Filed: Sep. 29, 2014

(51) Int. Cl. A61N 1/32; A61K 8/368; A61K 8/02; A61Q 7/02; A61Q 19/00; A61Q 19/02; A61Q 19/06; A61K 8/49; A61Q 19/08

(52) U.S. Cl. CPC A61N 1/325; A61K 8/4953; A61K 8/368; A61K 8/0212; A61Q 19/08; A61Q 19/007; A61Q 19/02; A61Q 19/06; A61Q 7/02; A61K 2800/10

(57) ABSTRACT

Cosmetic facial treatment delivery system with skincare benefit agents and iontophoresis micro-current zones are provided for delivering skincare treatments to multiple targeted areas of the skin to treat different concerns of an individual user. The system includes a sheet substrate with skincare agents and a reduction agent. An activating cream is applied to the skin along with the sheet substrate. The activating cream includes an oxidation agent. The combined reduction agent of the sheet substrate and the oxidation agent of the activating cream cause a redox reaction to generate micro-current. The micro-current generated facilitates delivery of skin benefit agents from the sheet by iontophoresis, or directly affects treatment by delivery of iontophoresis micro-current to the skin.

### FIELD OF THE INVENTION

The present invention relates to cosmetic treatment delivery sheets for achieving targeted and individualized delivery of one or more treatments to the skin of a user, in the form of a single-use sheet for delivering the skincare treatment to multiple targeted areas of the skin. In particular, the invention relates to targeted delivery of skincare treatments to areas of the facial skin of a user based on the unique skin profile of such user and to a cosmetic treatment delivery sheet mask which incorporates micro-current generating devices for application to and treatment of the targeted areas of the skin of the user by iontophoresis.

### BACKGROUND OF THE INVENTION

A variety of cosmetic patches or devices are commercially marketed or described as being useful for the delivery of skin treatment. It has been known to use cosmetic sheets comprised of various materials, such as non-woven cotton, elastically extendable or stretchable materials, thermoplastics, tacky gel, etc., impregnated with various cosmetic or dermatological preparations, for application to the skin of the face, the neck and other areas of the body. It is also know to use micro-current generating patches or masks. Currently, however, facial sheet masks and patches typically treat only one skin condition, either by applying a mask to the entire face or alternatively, a patch is applied only to certain areas, such as, under the eyes, to treat only this locus. However, these articles are inconvenient because, for example, they do not target one or more specific areas with one or more skin benefit agents, but only treat one local with one treatment. Most consumers have different concerns for their skin in different areas of their face. For example many consumers have combination skin in which the T-zone area (forehead, nose and chin) is oily while the remainder of the face is dry. For another example some consumers may have lines and wrinkles at the forehead, eye, and mouth areas, dry or flaky skin at the cheek areas, and hyperpigmentation spots at other areas. Each region would need different treatment products to address the different concerns. Conventional products typically only address one concern at a time by treating the entire facial skin, rather than only the targeted areas.

There is therefore a need for cosmetic treatment delivery sheet masks or patches which can deliver multiple treatments with micro-current to various targeted areas of the skin of a user to address different skin conditions of different areas of a user's face.

### SUMMARY OF THE INVENTION

Cosmetic treatment delivery sheets incorporating micro-current generators are provided for targeted and individualized skin treatment by iontophoresis of at least a first skin condition and a second skin condition in a predetermined treatment area of a user's skin. The delivery sheets have a sheet substrate arranged and constructed for conforming to the predetermined treatment area of the user's skin.

The substrate has defined thereon at least a first isolate, discrete region imprinted with a first skin benefit agent for treating the first skin condition. The first isolate, discrete region imprinted on the substrate in a first position corresponds to a first respective location of the first skin condition in the predetermined treatment area of the user's skin. The sheet also has a second isolate, discrete region imprinted with a second skin benefit agent for treating the second skin condition. The second isolate, discrete region is imprinted on the substrate in a second position corresponding to a second respective location of the second skin condition in the predetermined treatment area of the user's skin. The substrate, the first skin benefit agent or the second skin benefit agent may be water-soluble. At least one micro-current generator is secured to the sheet substrate. The micro-current generator comprises a power supply connected to a positive electrode and a negative electrode. The positive and negative electrodes are positioned on the substrate so as to deliver from the power supply micro-current to the user's skin in the predetermined treatment area in a quantity sufficient to facilitate delivery of at least one of the first skin benefit agent and the second skin benefit agent.

The skin benefit agents are selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents.

In an additional embodiment of the invention, the micro-current generators provide sufficient micro-current to treat the skin condition (i.e., with or without skin benefit agents).

Cosmetic sheets, including masks or patches, according to the present invention are provided with discrete regions, which are provided with one or more micro-current generators. Micro-current generators are in the form of a sheet or mask or patch that is powered by one or more thin film batteries or power sources. A micro-current generator may include a microprocessor for additional control of function such as, for example, current density profile or timing control. In addition to the power source or battery, each micro-current generator comprises two electrodes through which micro-current is supplied to the skin. The micro-current may be supplied to the skin through a medium or vehicle that includes bio-compatible salts to exhibit electromigration, electroosmosis and electrotransport of either charged or neutral skin benefiting agents into the skin. Each sheet, mask or patch according to the present invention treats multiple, different skin conditions of the predetermined treatment area by providing multiple isolate, discrete regions on the sheet, mask or patch, each with adapted to treat a different skin condition. The multiple isolate, discrete regions are adapted to treat different skin conditions by providing micro-current generators in the different regions to treat different conditions by, for example, programming each to produce a different micro-current power output for iontophoresis treatment. Alternatively, the sheet, mask or patch may be provided throughout with one micro-current power output level (through one or more micro-current generators), but is adapted to treat different skin conditions by being provided with different skin benefit agents in the multiple isolate, discrete regions, each skin benefit agent capable of treating a different skin condition. The micro-current generators and skin benefit agents may be different or the same in each region.

The micro-current generators and/or skin benefit agents may be provided to the delivery sheet in accordance with a skin profile generated by a skin analysis system such as those described in U.S. Pat. Nos. 8,425,477, 8,358,348 and 8,491,926, each incorporated herein by reference in their entirety.

Accordingly, when such cosmetic sheets, masks or patches are applied to and conformed to the skin, they can accurately deliver to the skin a pre-determined dosage of different skin care formulations for treating the skin chemically, or a predetermined dosage of micro-current to the skin for treating the skin electrically via iontophoresis, in each case for treating different skin conditions or providing different skin benefits. More preferably, the cosmetic sheets, masks or patches of the present invention may be specifically customized for individual users according to their unique skin profiles when combined with an imaging and analysis system for capturing an image of a predetermined treatment area of the user's skin, analyzing such data, and generating a skin profile indicative of the conditions of the predetermined treatment area of the user's skin. Additionally, a printing device may be communicatively connected with the system for printing one or more cosmetic delivery sheets, masks or patches, wherein each is arranged and constructed for conforming to a predetermined treatment area of a user's skin, and wherein each comprises a substrate with multiple isolate, discrete regions, wherein at least two of the isolate, discrete regions are imprinted with different skin benefit agents or provided or imprinted with different micro-current generators, for treating chemically or electrically different skin conditions of the predetermined treatment area according to the skin profile generated by the imaging and analysis system.

The present invention also relates to a cosmetic delivery sheet arranged and constructed for conforming to a predetermined treatment area of the skin of a user. Such a cosmetic delivery sheet includes a micro-current generator and a substrate with multiple isolate, discrete regions, wherein at least two of the isolate, discrete regions are imprinted with different skin benefit agents for treating different skin conditions of the predetermined treatment area.

The present invention in yet another aspect relates to a method for targeted and individualized delivery of multiple skin benefit agents to the skin of a user, which includes at least: (a) capturing an image of a predetermined treatment area of the user's skin; (b) analyzing the captured image data; (c) generating a skin profile indicative of the conditions of the predetermined treatment area of the user's skin; and (d) on one or more cosmetic delivery sheets including a micro-current generator, positioning the electrodes and/or printing skin treatment actives based on the generated skin profile, wherein the cosmetic delivery sheets are arranged and constructed for conforming to the predetermined treatment area of the user's skin, wherein each of the cosmetic delivery sheets comprises a micro-current generator and a substrate with multiple isolate, discrete regions, wherein at least two of the isolate, discrete regions are imprinted with different skin benefit agents for treating different skin conditions of the predetermined treatment area.

Other aspects and objectives of the present invention will become more apparent from the ensuing description, examples, and claims.

### BRIEF DESCRIPTION OF THE DRAWINGS

FIG. 1 is a schematic representation of a cosmetic treatment delivery sheet with a first substrate as a facial mask containing multiple isolate, discrete regions with different skin benefit agents, showing schematic location a of micro-current generator.

FIG. 2 is an exploded schematic representation of a cosmetic treatment delivery sheet with a first and second substrate as a facial mask containing multiple isolate, discrete regions with different skin benefit agents, showing schematic locations of micro-current generators.

FIG. 3 is an exploded schematic representation of a cosmetic treatment delivery sheet with a first and second substrate as a patch for the T-zone containing multiple isolate, discrete regions with different skin benefit agents and showing a schematic location of a micro-current generator.

FIG. 4 is an assembled schematic representation of a cosmetic treatment delivery sheet of FIG. 3 with a first and second substrate as a patch for the T-zone containing multiple isolate, discrete regions with different skin benefit agents and showing a schematic location of a micro-current generator.

FIG. 5 is an exploded schematic representation of a cosmetic treatment delivery sheet with a first and second substrate as a patch containing multiple isolate, discrete regions (for the crows foot region and under-eye region) with different skin benefit agents and showing a schematic location of a micro-current generator.

FIG. 6 is an assembled schematic representation of a cosmetic treatment delivery sheet of FIG. 5 with a first and second substrate as a patch containing multiple isolate, discrete regions (for the crows foot region and under-eye region) with different skin benefit agents and showing a schematic location of a micro-current generator.

FIG. 7 is an exploded schematic representation of a cosmetic treatment delivery sheet with a first and second substrate as a facial mask containing multiple isolate, discrete regions with different skin benefit agents, and a plurality of micro-current generators provided as a redox system, i.e., multiple oxidizing agents and corresponding reduction agents coupled by a hydrogel with salt.

FIG. 8 is an exploded schematic representation of a cosmetic treatment delivery sheet with a first and second substrate as a patch for the T-zone containing multiple isolate, discrete regions (for forehead and nose) with different skin benefit agents and showing a schematic location of a plurality of micro-current generators provided as a redox system, i.e., multiple oxidizing agents and corresponding reduction agents coupled by a hydrogel with salt.

FIG. 9 is an assembled schematic representation of a cosmetic treatment delivery sheet of FIG. 8 with a first and second substrate as a patch for the T-zone containing multiple isolate, discrete regions (for forehead and nose) with different skin benefit agents and showing a schematic location of a plurality of micro-current generators provided as a redox system, i.e., multiple oxidizing agents and corresponding reduction agents coupled by a hydrogel with salt.

FIG. 10 is an exploded schematic representation of a cosmetic treatment delivery sheet with a first and second substrate as a patch containing multiple isolate, discrete regions (for the crows foot region and under-eye region) with different skin benefit agents and showing a schematic location of a plurality of micro-current generators provided as a redox system, i.e., multiple oxidizing agents and corresponding reduction agents coupled by a hydrogel with salt.

FIG. 11 is an assembled schematic representation of a cosmetic treatment delivery sheet of FIG. 10 with a first and second substrate as a patch containing multiple isolate, discrete regions (for the crows foot region and under-eye region) with different skin benefit agents and showing a schematic location of a plurality of micro-current generators provided as a redox system, i.e., multiple oxidizing agents and corresponding reduction agents coupled by a hydrogel with salt.

### DETAILED DESCRIPTION OF THE INVENTION AND PREFERRED EMBODIMENTS THEREOF

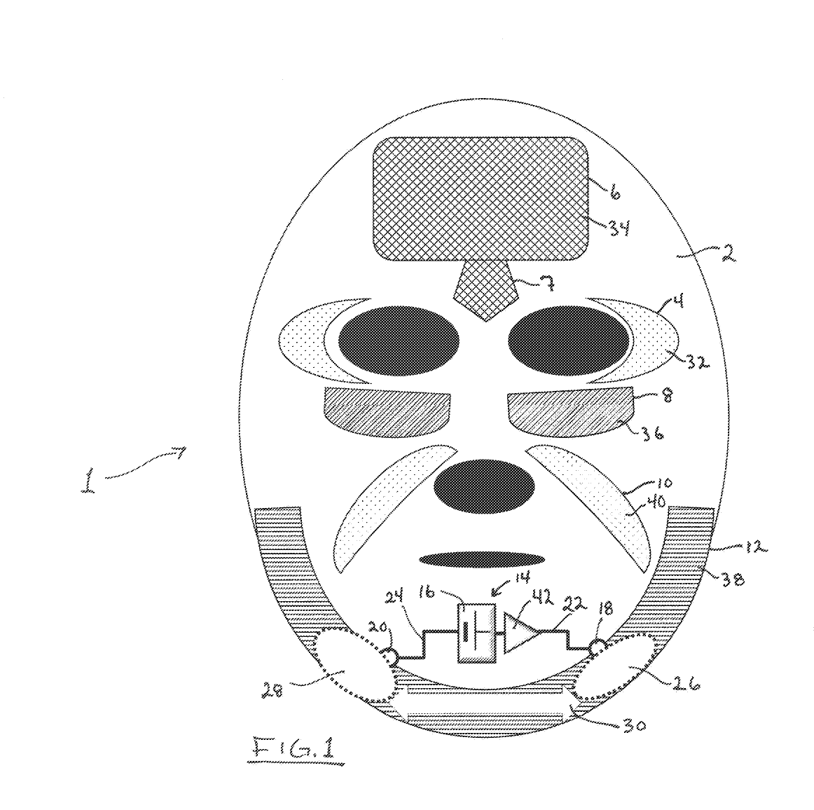

Referring now to FIG. 1, a first embodiment of a cosmetic treatment delivery sheet, shown generally at reference number 1, is provided for targeted and individualized skin treatment of at least a first skin condition and a second skin condition in a predetermined treatment area of a user's skin. The sheet has a first sheet substrate 2 arranged and constructed for conforming to the predetermined treatment area of the user's skin. As illustrated in FIG. 1, the substrate 2 is configured to be a full facial mask with apertures for the eyes, nose and mouth. The substrate has defined thereon at least a first isolate, discrete region 4 imprinted with a first skin benefit agent 32 for treating the first skin condition. The first isolate, discrete region 4 is imprinted on the substrate in a first position corresponding to a first respective location of the first skin condition in the predetermined treatment area of the user's skin. For example, the first region 4 in FIG. 1 corresponds to the crow's foot area adjacent the eye. A second isolate, discrete region 6 is provided on the substrate 4. The second isolate, discrete region 6 is imprinted with a second skin benefit agent 34 for treating the second skin condition. The second isolate, discrete region 6 is imprinted on the substrate in a second position corresponding to a second respective location of the second skin condition in the predetermined treatment area of the user's skin. For example, the second position in FIG. 1 corresponds to the forehead in the “T-zone”—the area above and between the eyes. Additional examples of isolate, discrete regions illustrated in FIG. 1 include, but are not limited to, the nose portion of the T-zone 7, the under-eye region 8, the “frown-line” region 10 and the chin region 12. Additional third, fourth and fifth skin benefit agents are illustrated schematically at reference numbers 36, 38 and 40, in the under-eye region, chin region and frown line region, respectively. The first sheet substrate is, generally speaking, a carrier for delivering the skin benefit agents to the appropriate treatment areas on the user's skin.

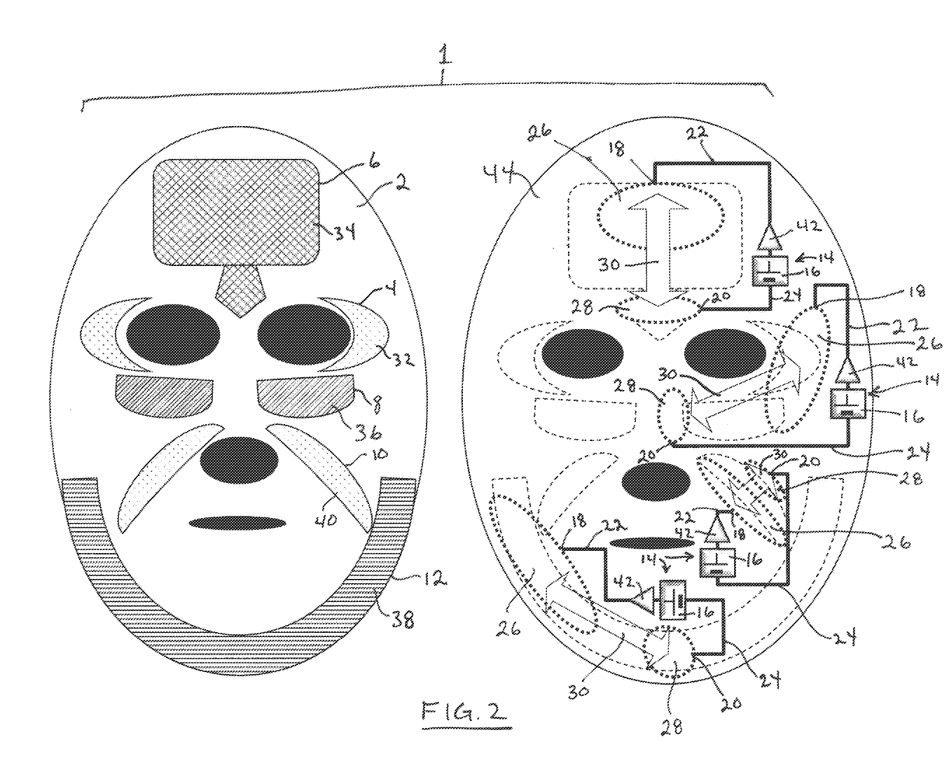

At least one micro-current generator 14 is secured to the sheet substrate 2. As illustrated, one micro-current generator is provided on the substrate 2, but the number of micro-current generators may be varied according to need. For example, as illustrated in FIG. 2, multiple micro-current generators may be provided to the cosmetic delivery sheet 1. Each micro-current generator has a power supply 16 connected by a positive circuit leg 22 to a positive electrode 18, and connected by a negative circuit leg 24 to a negative electrode 20. The positive electrode 18 and negative electrode 20 are positioned on the substrate 2 so as to deliver micro-current to the user's skin in the predetermined treatment area. The approximate area of micro-current delivery from positive electrodes 18 and negative electrodes 20 is illustrated schematically by bubbles 26 and 28, respectively. Arrow 30 indicates the approximate flow of the quantity of micro-current between the positive electrode 18 and negative electrode 20 that will take place after the delivery sheet is activated. Each micro-current generator 14 is adapted to deliver to the skin a quantity of micro-current sufficient to facilitate delivery of at least one of the skin benefit agents, 32, 34, 36, 38, 40, by iontophoresis. As illustrated in FIG. 1, the micro-current generator is configured to facilitate delivery of skin benefit agent 38 in the chin region 12, but it will be readily apparent that one or more micro-current generators may be added to the cosmetic treatment delivery sheet in any of the isolate, discrete regions to facilitate delivery of the other skin benefit agents, 32, 34, 36, 38, 40 etc., in one or multiple isolate, discrete regions, e.g. 4, 6, 7, 8, 12 or 10, respectively. Additionally, each micro-current generator 14 may be arranged to facilitate delivery of one skin benefit agent, or each micro-current generator 14 may be arranged to facilitate delivery of a plurality of skin benefit agents on the mask.

At least one of the isolate, discrete regions 4, 6, 7, 8, 10 or 12 may be imprinted with a skin benefit agent or agents according to a skin profile generated by a skin analysis system such as those described in U.S. Pat. Nos. 8,425,477, 8,358,348 and 8,491,926, each incorporated herein by reference in their entirety.

The positive electrode 18 or the negative electrode 20 may be positioned on the substrate 2 such that when the substrate 2 is applied to a treatment area on a user, the electrode 18, 20 contacts the user's skin directly.

Alternatively, a layer of bio-compatible salts may be provided on the substrate 2. The salts would be in contact with at least one of the positive or negative electrodes 18 or 20, and one of the skin benefit agents 32, 34, 36, 38, 40. The salts facilitate transport of the skin benefit agent 32, 34, 36, 38, 40 to the skin by one of electromigration, electroosmosis and electrotransport.

The substrate 2 and the skin benefit agents 32, 34, 36, 38, 40 may be water-soluble.

The skin benefit agents may be selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents.

Each isolate, discrete region may contain two or more different skin benefit agents. The skin benefit agents are preferably imprinted onto the substrate 2 as separate droplets without being mixed with one another.

The micro-current generator may further comprise a microprocessor 42. The microprocessor is preferably programmable to selectively control current delivery specifications such as the quantity of current, the current density profile and the timing, for example, of the treatment cycles. The micro-current delivery generator can be tuned with the onboard microprocessor to create the most favorable combination of current density and targeted skin benefiting agents to yield the best skin delivering action. For example, larger molecular weight skin benefit agents require higher current density as compared with smaller molecular weight agents. The cosmetic treatment delivery sheet may have the same printed composition of skin benefit agents, but delivery can be modified or enhanced by programming the microprocessor to yield different micro-current quantity or density or timing to change the amount and effectiveness of the skin benefit agent delivery.

Referring now to FIG. 2, shown is a second embodiment of the cosmetic treatment delivery sheet 1 having first and second substrates. Illustrated in FIG. 2 is an exploded schematic view of the treatment delivery sheet 1. A first sheet substrate 2 is arranged and constructed for conforming to the predetermined treatment area of the user's skin. The first sheet substrate 2 has defined thereon at least a first isolate, discrete region 4 imprinted with a first skin benefit agent 32 for treating the first skin condition. The first isolate, discrete region 4 is imprinted on the first sheet substrate 2 in a first position corresponding to a first respective location of the first skin condition in the predetermined treatment area of the user's skin. A second isolate, discrete region is 6, in the forehead portion of the T-zone, is imprinted with a second skin benefit agent 34 for treating the second skin condition. The second isolate, discrete region 6 is similarly imprinted on the first sheet substrate 2 in a second position corresponding to a second respective location of the second skin condition in the predetermined treatment area of the user's skin. The first sheet substrate 2 has additional isolate, discrete regions illustrated at the nose portion 7 of the T-zone, the under-eye regions 8, the ‘frown-line’ regions 10 and the chin region 12. Additional skin benefit agents are illustrated schematically at reference numbers 36, 38 and 40, in the under-eye region, chin region and frown line region, respectively.

A second sheet substrate 44 is provided corresponding in shape and arrangement to the first sheet substrate 2. The second sheet substrate 44 may alternatively be referred to as the micro-current delivery backing sheet. To form the cosmetic treatment delivery sheet 1, the first sheet substrate 2 is secured to the second sheet substrate 44 such that the isolate discrete regions on the first substrate sheet 2 line up with the corresponding broken line depictions of those regions on the second sheet substrate 44. A micro-current generator 14 is secured to the second sheet substrate 44. The micro-current generator has a power supply 16 connected to a positive electrode 18 by a positive circuit leg 22. The power supply 16 is connected to a negative electrode 20 by a negative circuit leg 24. The positive electrode 18 and negative electrode 20 are positioned on the second sheet substrate 44 such that micro-current can be delivered to the user's skin in the predetermined treatment area in a quantity sufficient to facilitate delivery of at least one of the first skin benefit agent 32 and the second skin benefit agent 34 by iontophoresis. The quantity of micro-current is preferably delivered through at least one of the isolate, discrete regions of the first sheet substrate 2, e.g., the first isolate, discrete region 4 or the second isolate, discrete region 6, etc. The approximate area of micro-current delivery from positive electrodes 18 and negative electrodes 20 is illustrated schematically by broken line bubbles 26 and 28, respectively. Arrow 30 indicates the approximate flow of the quantity of micro-current between the positive electrode 18 and negative electrode 20 that will take place after the delivery sheet is activated.

At least one of the first isolate, discrete region 4 and the second isolate, discrete region 6 may be imprinted according to a skin profile generated by a skin analysis system as described, for example in U.S. Pat. No. 8,358,348 B2, incorporated herein by reference.

The positive electrode 18 and/or the negative electrode 20 may be positioned on the second sheet substrate 44 such that when the cosmetic treatment delivery sheet 1, i.e., the first sheet substrate 2 attached to the second sheet substrate 44, is applied to the predetermined treatment area, one or both electrodes contact the user's skin directly.

A layer of bio-compatible salts may be provided on the first sheet substrate 2 or the second sheet substrate 44. The layer of bio-compatible salts may be provided such that it is in contact with at least one of the positive electrode 18 and the negative electrode 20, as well as a corresponding one of the skin benefit agents 32, 34, 36, 38 or 40. The salts are selected and provided so as to facilitate transport of a corresponding one of the skin benefit agents to the skin by one of electromigration, electroosmosis and electrotransport.

At least one of the first sheet substrate 2, the skin benefit agents 32, 34, 36, 38, and 40 may be water-soluble. Alternatively, the substrate 2 may be water insoluble. The second sheet substrate supporting the micro-current generator or multiple micro-current generators may be reusable by, for example, being made of a water insoluble material upon which a fresh first sheet substrate with a new supply of skin benefit agents may be secured after each use. The second sheet substrate may be made of a memory foam or comprise a memory foam mask upon which the first sheet substrate with skin benefit agents is secured. Such a memory foam mask is commercially available from TechNature company. In this way, complex, customizable skincare treatments can be created by printing skincare benefit agents on substrates with customizable or programmable delivery with built-in micro-current technology.

The micro-current technology is preferably a sheet/mask/patch that is powered by thin film battery or power source, either with or without a microprocessor for additional control on current density profile and timing.

The skin benefit agents are selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents. The skin benefit agents may be either charged or neutral.

Each isolate, discrete region may contain two or more different skin benefit agents. The different skin benefit agents may be imprinted onto the first sheet substrate as separate droplets without being mixed with one another.

Micro-current generators 14 may be provided to each isolate, discrete region. For example, a micro-current generator may be provided to one or more of the crow's foot region 4, the forehead portion of the T-zone 6, the nose portion of the T-zone 7, the under-eye region, the frown line region or the chin region.

Each micro-current generator 14 may further comprise a microprocessor 42. The microprocessor is preferably programmable by known means to selectively control at least one of current density profile and timing.

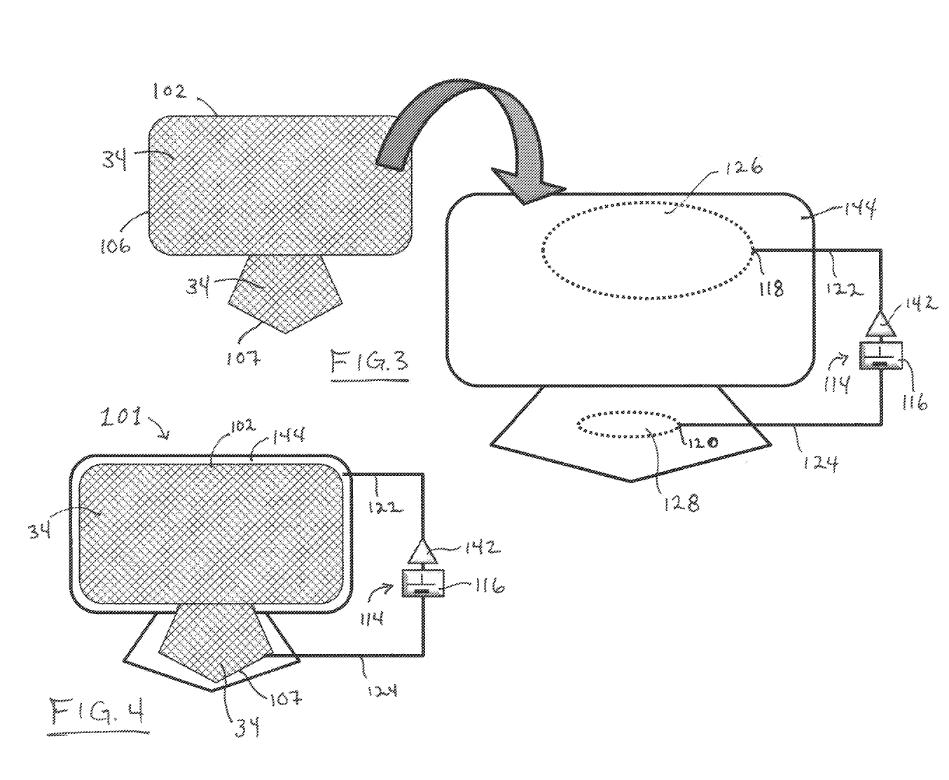

FIGS. 3 and 4 illustrate a third embodiment of the cosmetic treatment delivery sheet in the form of a sheet or patch specifically for targeting the T-zone. FIG. 3 is an exploded schematic representation of the T-zone cosmetic treatment delivery sheet, and FIG. 4 illustrates the assembled cosmetic treatment delivery sheet 101 for the T-zone. A first sheet substrate 102 is arranged and constructed for conforming to the predetermined treatment area of the user's skin in the T-zone. The first sheet substrate 102 has defined thereon at least a first isolate, discrete region 106 imprinted with the skin benefit agent 34 for treating the first skin condition. The first isolate, discrete region 106 is imprinted on the first sheet substrate 102 in a first position corresponding to a first respective location of the first skin condition in the predetermined treatment area of the user's skin, i.e., the forehead portion of the T-zone. A second isolate, discrete region 107 is imprinted with the skin benefit agent 34 for treating the second skin condition. The second isolate, discrete region 107 is imprinted on the first sheet substrate 202 in a second position corresponding to a second respective location of the second skin condition in the predetermined treatment area of the user's skin, i.e., the nose portion of the T-zone.

A second sheet substrate 144 is provided corresponding generally in shape and arrangement to the first sheet substrate 102. To form the cosmetic treatment delivery sheet 101 shown in FIG. 4, the first sheet substrate 102 is secured to the second sheet substrate 144 such that the isolate discrete regions on the first substrate sheet 102 line up with the corresponding broken line depictions of those regions on the second sheet substrate 144. A micro-current generator 114 is secured to the second sheet substrate 144. The micro-current generator has a power supply 116 connected to a positive electrode 118 by a positive circuit leg 122. The power supply 116 is connected to a negative electrode 120 by a negative circuit leg 124. The positive electrode 118 and negative electrode 120 are positioned on the second sheet substrate 144 such that micro-current can be delivered to the user's skin in the predetermined treatment area in a quantity sufficient to facilitate delivery of the skin benefit agent 34 by iontophoresis. The quantity of micro-current is preferably delivered through at least one of the isolate, discrete regions of the first sheet substrate 102, e.g., the isolate, discrete region 106 or the isolate, discrete region 107, etc., via positive delivery area 126 and negative delivery area 128. The micro-current generator 114 may further comprise a microprocessor 142. The microprocessor is preferably programmable by known means to selectively control at least one of current density profile and timing. At least one of the first isolate, discrete region 106 and the second isolate, discrete region 107 may be imprinted according to a skin profile generated by a skin analysis system as described, for example in U.S. Pat. No. 8,358,348 B2, incorporated herein by reference. The positive electrode 118 and/or the negative electrode 120 may be positioned on the second sheet substrate 144 such that when the cosmetic treatment delivery sheet 101, i.e., the first sheet substrate 102 attached to the second sheet substrate 144, is applied to the predetermined treatment area, one or both electrodes contact the user's skin directly. A layer of bio-compatible salts may be provided on the first sheet substrate 102 or the second sheet substrate 144. The layer of bio-compatible salts may be provided such that it is in contact with at least one of the positive electrode 118 and the negative electrode 120, as well as the skin benefit agent 34. The salts are selected and provided so as to facilitate transport of a corresponding one of the skin benefit agents to the skin by one of electromigration, electroosmosis and electrotransport. At least one of the first sheet substrate 102 and the skin benefit agent 34 may be water-soluble. The skin benefit agent 34 is selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents.

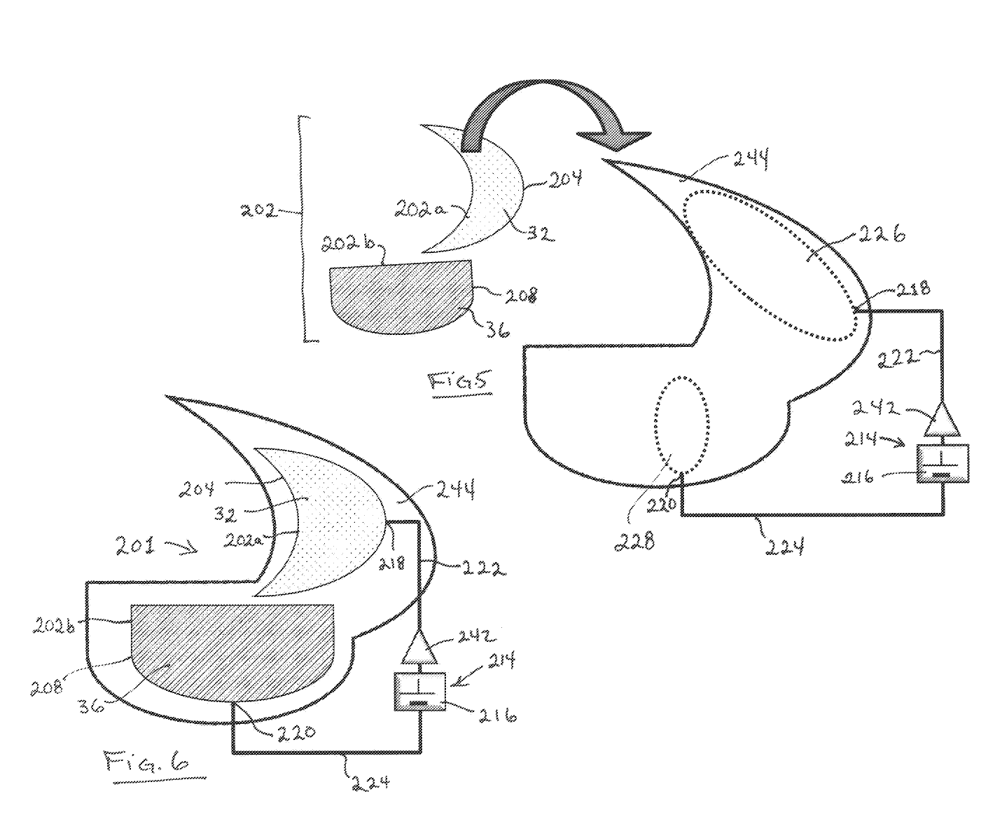

FIGS. 5 and 6 illustrate a fourth embodiment of the cosmetic treatment delivery sheet in the form of a sheet or patch specifically for targeting the crow's foot zone and the under-eye zone. FIG. 5 is an exploded schematic representation of the crow's foot/under-eye cosmetic treatment delivery sheet, and FIG. 6 illustrates the assembled cosmetic treatment delivery sheet 201 for the crow's foot zone and under-eye zone. A first sheet substrate 202, illustrated as separate portions 202a and 202b, is arranged and constructed for conforming to the predetermined treatment area of the user's skin in the T-zone. The first sheet substrate 202 has defined thereon at least a first isolate, discrete region 204 imprinted with the skin benefit agent 32 for treating the first skin condition. The first isolate, discrete region 204 is imprinted on the first sheet substrate 202 in a first position corresponding to a first respective location of the first skin condition in the predetermined treatment area of the user's skin, i.e., the crow's foot zone. A second isolate, discrete region 208 is imprinted with the skin benefit agent 36 for treating the second skin condition. The second isolate, discrete region 208 is imprinted on the first sheet substrate 202 in a second position corresponding to a second respective location of the second skin condition in the predetermined treatment area of the user's skin, i.e., the under-eye zone.

A second sheet substrate 244 is provided corresponding generally in shape and arrangement to the first sheet substrate 202. To form the cosmetic treatment delivery sheet 201 shown in FIG. 6, the first sheet substrate 202 is secured to the second sheet substrate 244 such that the isolate discrete regions on the first substrate sheet 202 line up with the corresponding broken line depictions of those regions on the second sheet substrate 244. A micro-current generator 214 is secured to the second sheet substrate 244. The micro-current generator has a power supply 216 connected to a positive electrode 218 by a positive circuit leg 222. The power supply 216 is connected to a negative electrode 220 by a negative circuit leg 224. The positive electrode 218 and negative electrode 220 are positioned on the second sheet substrate 244 such that micro-current can be delivered to the user's skin in the predetermined treatment area in a quantity sufficient to facilitate delivery of the skin benefit agent 32 and or skin benefit agent 36 by iontophoresis. The quantity of micro-current is preferably delivered through at least one of the isolate, discrete regions of the first sheet substrate 202, e.g., the isolate, discrete region 204 or the isolate, discrete region 208, etc., via positive delivery area 226 and negative delivery area 228. The micro-current generator 214 may further comprise a microprocessor 242. The microprocessor is preferably programmable by known means to selectively control at least one of current density profile and timing. At least one of the first isolate, discrete region 204 and the second isolate, discrete region 208 may be imprinted according to a skin profile generated by a skin analysis system as described, for example in U.S. Pat. No. 8,358,348 B2, incorporated herein by reference. The positive electrode 218 and/or the negative electrode 220 may be positioned on the second sheet substrate 244 such that when the cosmetic treatment delivery sheet 201, i.e., the first sheet substrate 202 attached to the second sheet substrate 244, is applied to the predetermined treatment area, one or both electrodes contact the user's skin directly. A layer of bio-compatible salts may be provided on the first sheet substrate 202 or the second sheet substrate 244. The layer of bio-compatible salts may be provided such that it is in contact with at least one of the positive electrode 218 and the negative electrode 220, as well as the skin benefit agents 32, 36. The salts are selected and provided so as to facilitate transport of a corresponding one of the skin benefit agents to the skin by one of electromigration, electroosmosis and electrotransport. At least one of the first sheet substrate 202 and the skin benefit agents 32, 36 may be water-soluble. The skin benefit agents 32, 36 are selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents.

The power supply, generally speaking, may take the form of a battery, a capacitor, a solar cell, a connection to an electric grid, or any other suitable source of micro-current power.

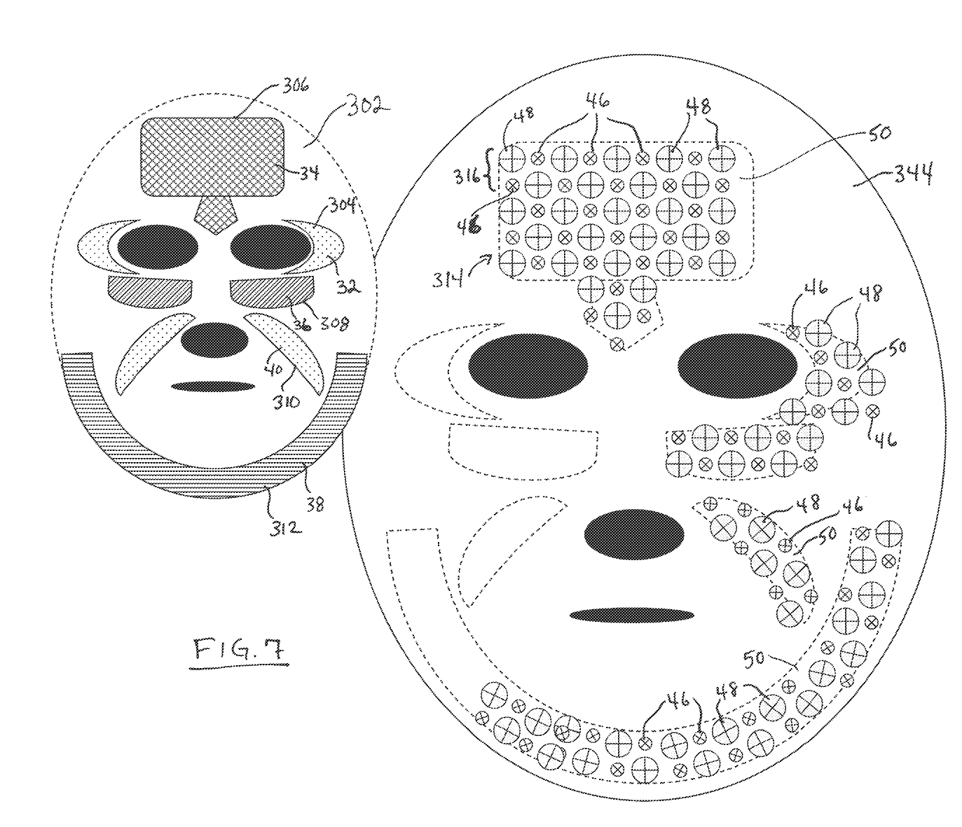

Alternatively, as illustrated in FIGS. 7-11, the power supply 316 for the micro-current generator 314 may comprise components for a redox reaction, i.e., an oxidation agent 46, a reduction agent 48 and a hydrogel with salt 50 adapted to trigger micro-current flow from the generator 316. This power supply 316 is a printed electrochemical cell, alternatively referred to herein as an “imprint battery”. Referring to FIG. 7, the imprint battery can be made by printing conducting electrode contact points with spontaneous oxidation-reduction agents in different spots adjacent to each other on the second sheet substrate 344, securing the second sheet substrate 344 to the first sheet substrate 302 having skin benefit agent 32 (and/or others), and applying the combined sheet substrates to the treatment area(s) with an appropriate medium (hydrogel with salt) to trigger the micro-current on the skin. Both skin benefit agents and electrochemical cells can be customized and printed with existing printing technology based on individual users' different needs. This electrochemical cell (galvanic cell) type of cosmetic treatment delivery sheet in patch or mask form provides micro-current to enhance delivery of skin benefit agents by spontaneous oxidation-reduction reaction of reducing and oxidizing agents. The cosmetic treatment delivery sheet can be customized either by customizing the skin benefit agents on the sheet or by customizing the micro-current fields. The agents can be customized, for example, in accordance with a skin profile generated by a skin analysis system, i.e., based on individual needs and location of skin concerns. Each skin benefit agent can be programmed to be deposited by printer at different locations, in dots per inch deposition density and various combinations with other skin benefit agents. The micro-current can be customized by selecting reducing and oxidizing agents such as, for example, silver or zinc, in an amount or weight ratio between two agents and with spacing distances, to alter the strength of the micro-current delivered, thus meeting the individual skin treatment needs.

In greater detail, the fifth embodiment illustrated in FIG. 7 has a first sheet substrate 302 (not to scale) arranged and constructed for conforming to the predetermined treatment area of the user's skin. The first sheet substrate 302 has defined thereon at least a first isolate, discrete region 304 imprinted with a first skin benefit agent 32 for treating the first skin condition. The first isolate, discrete region 304 is imprinted on the first sheet substrate 302 in a first position corresponding to a first respective location of the first skin condition in the predetermined treatment area of the user's skin. A second isolate, discrete region is 306, in the forehead portion of the T-zone, is imprinted with a second skin benefit agent 34 for treating the second skin condition. The second isolate, discrete region 306 is imprinted on the first sheet substrate 302 in a second position corresponding to a second respective location of the second skin condition in the predetermined treatment area of the user's skin. The first sheet substrate 302 has additional isolate, discrete regions illustrated at the nose portion 307 of the T-zone, the under-eye regions 308, the ‘frown-line’ regions 310 and the chin region 312. Additional skin benefit agents are illustrated schematically at reference numbers 36, 38 and 40, in the under-eye region 308, chin region 312 and frown line region 310, respectively.

A second sheet substrate 344 is provided corresponding in shape and arrangement to the first sheet substrate 302. To form the cosmetic treatment delivery sheet 301, the first sheet substrate 302 is secured to the second sheet substrate 344 such that the isolate discrete regions on the first substrate sheet 302 line up with the corresponding broken line depictions of those regions on the second sheet substrate 344. The micro-current generator 314 in the form of redox reaction components, oxidation agent 46, reduction agent 48 and hydrogel 50, is printed or otherwise secured to the second sheet substrate 344. As illustrated in FIG. 7 by broken lines, the hydrogel applications closely approximate the size, shape and area of the various isolate, discrete regions 304, 306, 308, 310, 312 (e.g., crow's foot, t-zone, under-eye region, chin region, etc.). As the hydrogel 50 is adapted to trigger the redox reaction between the oxidation agent 46 and the reduction agent 48 to trigger the redox reaction to generate the micro-current, the hydrogel 50 may be separately provided to be applied either to the cosmetic delivery sheet 301 or to the user's skin just prior to application of the sheet 301. The hydrogel may be applied proximal to and in contact with the oxidation agent and the reduction agent. Alternatively, the hydrogel 50 may be provided in an inactive form that is activated, for example, upon wetting with water or another suitable solvent. Upon activation by the hydrogel 50, the oxidation agent 46 and reduction agent 48, or multiple oxidation agents 46 and corresponding reduction agents 48, generate a quantity of micro-current via a redox reaction. The oxidation agent 46 and reduction agent 48, or multiple oxidation agents 46 and corresponding reduction agents 48, are positioned on the second sheet substrate 344 such that micro-current that is generated can be delivered to the user's skin in the predetermined treatment area in a quantity sufficient to facilitate delivery of at least one of the first skin benefit agents 32, 34, 36, 38 or 40 by iontophoresis. The quantity of micro-current is preferably delivered through at least one of the isolate, discrete regions of the first sheet substrate 302, e.g., isolate, discrete regions 304, 306, 308, 310, 312, etc.

The cosmetic treatment delivery sheet 301 may be provided with one or more skin benefit agents according to a skin profile generated by a skin analysis system.

The cosmetic treatment delivery sheet 301 may include a layer of bio-compatible salts provided on the substrate in contact with the micro-current generator redox system. The salts are adapted to facilitate transport of the quantity of micro-current to the skin by one of electromigration, electroosmosis and electrotransport.

The skin benefit agents for the cosmetic treatment delivery sheet 301 may be selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents. The different skin benefit agents may be imprinted onto the first sheet substrate 302 as separate droplets without being mixed with one another.

The first sheet substrate 302, the second sheet substrate 344, the skin benefit agents 332, 334, 336, 338, 340 and the redox reaction components, oxidation agent 46, reduction agent 48 and hydrogel 50 may all be water-soluble. Accordingly, one advantage of powering the cosmetic delivery sheet 301 with the redox reaction components 46, 48, 50 is that the entire system and all of its components may be water soluble. This may be a more preferred embodiment for consumers as the entire mask can be rinsed away after the skin benefit agents are absorbed sufficiently into the skin.

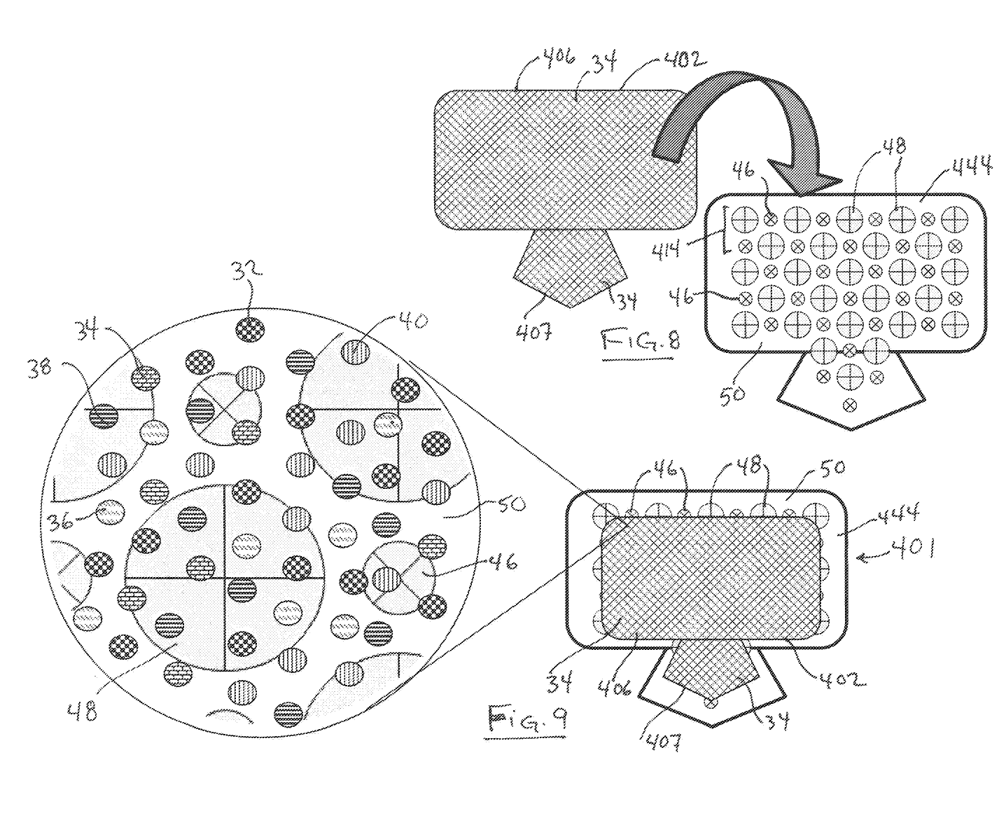

FIGS. 8 and 9 illustrate a sixth embodiment 401 of the cosmetic treatment delivery sheet in the form of a sheet or patch using a redox reaction system for micro-current and specifically for targeting the T-zone. The sixth embodiment 401 functions as described above for the fifth embodiment 301. FIG. 8 is an exploded schematic representation of the T-zone cosmetic treatment delivery sheet, and FIG. 9 illustrates the assembled cosmetic treatment delivery sheet 401 for the T-zone. A first sheet substrate 402 is arranged and constructed for conforming to the predetermined treatment area of the user's skin in the T-zone. The first sheet substrate 402 has defined thereon at least a first isolate, discrete region 406 imprinted with the skin benefit agent 34 for treating the first skin condition. The first isolate, discrete region 406 is imprinted on the first sheet substrate 402 in a first position corresponding to a first respective location of the first skin condition in the predetermined treatment area of the user's skin, i.e., the forehead portion of the T-zone. A second isolate, discrete region 407 is imprinted with the same skin benefit agent 34 for treating the second skin condition. The second isolate, discrete region 407 is imprinted on the first sheet substrate 402 in a second position corresponding to a second respective location of the second skin condition in the predetermined treatment area of the user's skin, i.e., the nose portion of the T-zone.

A second sheet substrate 444 is provided corresponding generally in shape and arrangement to the first sheet substrate 402. To form the cosmetic treatment delivery sheet 401 shown in FIG. 9, the first sheet substrate 402 is secured to the second sheet substrate 444 such that the isolate discrete regions on the first substrate sheet 402 line up with the second sheet substrate 444. A micro-current generator 414 in the form of redox reaction components, oxidation agent 46, reduction agent 48 and hydrogel 50, is printed or otherwise secured to the second sheet substrate 444. Upon activation by the hydrogel 50, the oxidation agent 46 and reduction agent 48, or multiple oxidation agents 46 and corresponding reduction agents 48, generate a quantity of micro-current via a redox reaction. The oxidation agent 46 and reduction agent 48, or multiple oxidation agents 46 and corresponding reduction agents 48, are positioned on the second sheet substrate 444 such that micro-current that is generated can be delivered to the user's skin in the predetermined treatment area in a quantity sufficient to facilitate delivery of at least one of the skin benefit agents 32, 34, 36, 38 or 40 by iontophoresis. The quantity of micro-current is preferably delivered through at least one of the isolate, discrete regions of the first sheet substrate 402, e.g., isolate, discrete regions 306 or 307. The sixth embodiment 401 functions as described above for the fifth embodiment 301, and may include the additional, optional features described above, e.g., embodiment 401 may be water soluble, may have skin benefit agents according to a skin profile, may have bio-compatible salts, etc.

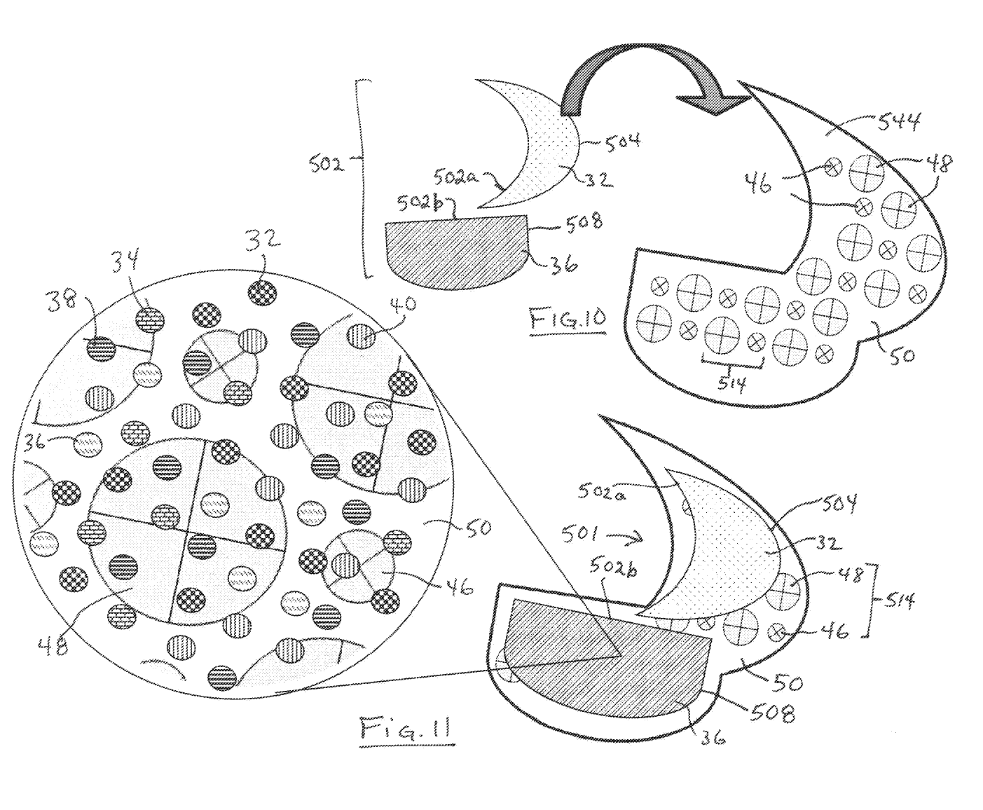

FIGS. 10 and 11 illustrate a seventh embodiment 501 of the cosmetic treatment delivery sheet in the form of a sheet or patch using a redox reaction system for micro-current and specifically for targeting the crow's foot zone and the under-eye zone. The seventh embodiment 501 functions as described above for the fifth embodiment 301. FIG. 10 is an exploded schematic representation of the crow's foot zone and the under-eye zone cosmetic treatment delivery sheet, and FIG. 9 illustrates the assembled cosmetic treatment delivery sheet 501 for the crow's foot zone and the under-eye zone. A first sheet substrate 502, illustrated in two portions as 502a and 502b, is arranged and constructed for conforming to the predetermined treatment area of the user's skin in the crow's foot zone and the under-eye zone, respectively. The first sheet substrate 502 has defined thereon at least a first isolate, discrete region 504 imprinted with the skin benefit agent 32 for treating the first skin condition. The first isolate, discrete region 504 is imprinted on the first sheet substrate 502 in a first position corresponding to a first respective location of the first skin condition in the predetermined treatment area of the user's skin, i.e., the forehead portion of the T-zone. A second isolate, discrete region 508 is imprinted with the skin benefit agent 36 for treating the second skin condition. The second isolate, discrete region 508 is imprinted on the first sheet substrate 502 in a second position corresponding to a second respective location of the second skin condition in the predetermined treatment area of the user's skin, i.e., the nose portion of the T-zone.

A second sheet substrate 544 is provided corresponding generally in shape and arrangement to the first sheet substrate 502, portions 502a and 502b. To form the cosmetic treatment delivery sheet 501 shown in FIG. 9, the first sheet substrate 502, including portions 502a and 502b, is secured to the second sheet substrate 544 such that the isolate discrete regions on the first substrate sheet 502 line up with the second sheet substrate 544. A micro-current generator 514 in the form of redox reaction components, oxidation agent 46, reduction agent 48 and hydrogel 50, is printed or otherwise secured to the second sheet substrate 544. Upon activation by the hydrogel 50, the oxidation agent 46 and reduction agent 48, or multiple oxidation agents 46 and corresponding reduction agents 48, generate a quantity of micro-current via a redox reaction. The oxidation agent 46 and reduction agent 48, or multiple oxidation agents 46 and corresponding reduction agents 48, are positioned on the second sheet substrate 544 such that micro-current that is generated can be delivered to the user's skin in the predetermined treatment area in a quantity sufficient to facilitate delivery of at least one of the skin benefit agents 32, 36 by iontophoresis. The quantity of micro-current is preferably delivered through at least one of the isolate, discrete regions of the first sheet substrate 502, e.g., isolate, discrete regions 504 or 508. The seventh embodiment 501 functions as described above for the fifth embodiment 301, and may include the additional, optional features described above, e.g., embodiment 501 may be water soluble, may have skin benefit agents according to a skin profile, may have bio-compatible salts, etc.

Optionally, the cosmetic treatment delivery sheet 1, 101, 201, 301, 401 or 501 may have multiple skin benefit agents in each isolate, discrete region. This is shown for illustrative purposes in FIGS. 9 and 11 wherein an enlarged view of the isolate discrete regions 406 and 508, respectively, includes skin benefit agents 32, 34, 36, 38 and 40. The skin benefit agents may be selected from the group consisting of: (1) anti-wrinkle or skin-tightening agents; (2) anti-aging agents; (3) moisturizing agents; (4) skin whitening or depigmentation agents; (5) anti-inflammatory agents; (6) anti-acne agents; (7) DNA repair agents; (8) skin lipid barrier repair agents; (9) anti-cellulite agents; (10) wound-healing agents; (11) stretch-mark/scar removing agents; (12) plumping agents; (13) hair growth retardation agents; (13) hair growth stimulating agents; (14) dark cycle reduction or de-puffing agents; (15) collagen synthesis or blood circulation enhancing agents; (16) antioxidants; (17) sebum-controlling agents; and (18) pore-minimizing agents. The different skin benefit agents may be imprinted onto the first sheet substrate as separate droplets without being mixed with one another.

Suitable thin film batteries or similar power sources should deliver a quantity of micro-current in the range of 1.5 volts to 3.0 volts, in a current density of 0.1 mA/cm² and capacity of 3 to 90 mAh. Such batteries and power sources are commercially available from companies such as Blue Spark Technology, Enfucell and Power Paper. Alternate power sources may be used. These alternate power sources include rechargeable power source such as a mobile device, a personal electronic device, a stand-alone rechargeable battery, a capacitor or a solar panel power generator. These alternatives would be separate units from the cosmetic treatment delivery sheet that are connected to the sheet via a cable or induction circuits. Accordingly, the cosmetic treatment delivery sheet in any form (i.e., mask, patch, etc.) may be reusable and/or have a rechargeable power source.

Alternatively, either a cathode or anode can be imprinted on the second sheet substrate, and the other of the cathode or anode can be incorporated in an activating cream. Accordingly, only one of the reducing agent or the oxidizing agent is imprinted on the second sheet substrate, and the other of the reducing agent or the oxidizing agent is provided in the form of an activating cream such as a hydrogel. When the cream and the substrate are combined on the skin, the electrochemical cell mechanism is completed and micro-current is generated on the skin.

As yet another alternative, a ‘battery’ cream may be provided that includes two metals with different charges in a dual phase product. The electrochemical cell mechanism may be prepared by applying a dual phase product. A first phase contains the reduction agent and a second phase contains the oxidation agent. Upon combination of the first and second phase creams and application to the face along with the first sheet substrate with skin benefit agents (either dissolvable or non-dissolvable), the electrochemical cell mechanism is triggered and micro-current is provided to enhance or modify the delivery of the skin benefit agents on the first sheet substrate.

The above systems provide personalized micro-current fields based on each person's skin concerns and needs in sheet, mask or patch form. The advantage is to have a skincare treatment with both personalized skincare benefit agents and customized iontophoresis micro-current zones to treat different concerns of each individual user. Current existing iontophoresis patches can only deliver one composition for skin treatment at a time. Current patches cannot provide targeted and individualized skincare treatments, plus personalized micro-current fields based on each person's skin concerns and needs. Examples of concerns that can be addressed are: lines and wrinkles, dark circles, age spot, acne, sagginess, evenness of skin tone, cellulite, spider vein, lifting, Rosacea, dryness, etc.

The foregoing is achieved by printing skin benefit agents on a dissolvable or non-dissolvable substrate (first sheet substrate 1, 101, 201, 301, 401, 501) and then attaching the substrate to a micro-current generator backing sheet (second substrate 44, 144, 244, 344, 444, 544). Alternatively, it is achieved by printing skin benefit agents on a dissolvable or non-dissolvable substrate (first sheet substrate 1, 101, 201, 301, 401, 501) and then attaching the substrate to a memory foam mask with micro-current generator(s) powered by a rechargeable battery power source (such as a mobile device). The mask is reusable. As yet another alternative, it is achieved by printing skin benefit agents on a dissolvable or non-dissolvable substrate (first sheet substrate 1, 101, 201, 301, 401, 501) and then printing micro-current generators with power sources in different locations directly on the first sheet substrate. As yet another alternative, it is achieved by printing a cathode or anode on a dissolvable or non-dissolvable substrate (first sheet substrate 1, 101, 201, 301, 401, 501) and then incorporating the other of the cathode or anode in an activating cream to be applied to or with the substrate to activate the micro-current. As yet another alternative, it can be achieved by preparing a two phase battery cream with a different metal in each phase and combining the two phases and applying to the skin to generate a micro-current.

The printing of skin benefit agents onto the first sheet substrate is described in more detail as follows. A computerized or computer-aided system may be used for achieving the targeted and individualized delivery of multiple skin benefit agents to the skin of a user based on the unique skin profile of the user. Cosmetic delivery products produced by the system of the present invention are capable of delivering multiple skin benefit agents to multiple target sections or regions on demand and according to the user skin profile with precise dosage and location control.

Preferably, such a system includes at least an imaging device for capturing an image of the desired treatment area of the user's skin. Such desired treatment skin area may be, for example, full face, partial face, neck, thigh, or the like. In a particularly preferred but not necessary embodiment of the present invention, the desired treatment area is the full face of the user. The imaging device is preferably a digital camera, which may capture the images of the desired treatment area in conjunction with a light source that delivers sufficient and consistent visible or invisible light, such as infrared light or near infrared light. The imaging device can be set in either a manual or an automatic mode for identifying the desired treatment area.

The captured images are directly converted by such imaging device into digital data and stored therein or sent to a personal computer or other computerized analyzing device that is communicatively connected with the imaging device. The analyzing device is programmed for analyzing image data and generating a skin profile indicative of the conditions of the desired treatment area of the user's skin based on the image data. Preferably, the skin profile defines skin regions with certain defects or concerns that need certain types of treatment. The term “defects” and/or “concerns” as used herein broadly covers any types of sub-optimal skin conditions, either perceived or otherwise, such as skin dryness, flakiness, redness, oiliness, large pores, dullness, dark spots, uneven skin tone, acne scars, fine lines and wrinkles, under-eye dark circles, under-eye puffiness, cellulite, and the like, or any types of abnormal skin conditions or disorders. More preferably, the skin profile also defines the severity of the skin defects. Such skin profile can be generated using various known algorithms. Examples of these algorithms are described in greater detail by Japanese Patent Application Publication No. 95-231883 entitled “Skin Surface Analysis System and Skin Surface Analysis Method”; International Patent Application Publication No. WO98/37811 entitled “Systems and Methods for the Multispectral Imaging and Characterization of Skin Tissue”; and U.S. Pat. No. 5,016,173 entitled “Apparatus and Method for Monitoring Visually Accessible Surfaces of the Body,” the contents of which are incorporated in their entireties for all purposes. Commercially available skin imaging tools that can be used for diagnosing skin defects in the present invention include, for example, the VISIA® Complexion Analysis tools available from Canfield Scientific, Inc. (Fairfield, N.J.), thermal camera system, laser Doppler imaging system, translucency meter, mexameter, Mexameter® MX18 available from CK Electronic (Koln, Germany), the CR series Chroma Meters available from Konica Minolta Business Solutions, U.S.A. (Ramsey, N.J.), the SIAMETRICS™ and COSMETRICS™ skin visualization and measurement systems available from Astron Clinica Ltd. (Cambridge, United Kingdoms), and the like. If the severity of the skin defects is represented by a numerical value, it may be desirable to normalize such value based on the user's ethnic origin, age, geographic location, or any other factor that may have an impact on the user's skin conditions.

Once the skin profile is generated, it is processed by well known photo-editing and illustration software programs, such as Adobe Photoshop Element 4.0, Microsoft PowerPoint 2003, and the like, for creating images to be outputted to a printing device, which corresponding print out one or more cosmetic delivery sheets that are customized for the user based on his or her unique skin conditions. Preferably, the printing device is a printer that contains multiple cartridges, each of which is filled with a composition containing one or more skin benefit agents. Because the conventional thermal inkjet printing mechanism produce high temperature environment at the print head during ink discharging step, which may degrade or destabilize certain skin benefit agents, it is preferred that the printing in the present invention is carried out using a heatless printing mechanism. For example, a pressure-driven ink jet can be used, in which pressure is created on demand by a piezoelectric transducer to change the shape of an internal diaphragm in the inkjet print head and therefore force droplets of the skin benefit agents contained in the ink tank to be deposited onto the substrate. Suitable printing devices for the practice of the present invention include, for example, the Epson Workforce series, preferably Epson Workforce 30, the Spectra piezoelectric printers from Fujifilm Dimatix, the RISO HC5500 inkjet printer, and the like. Alternatively, when the skin benefit agents to be delivered are thermally stable or relatively less susceptible to thermal degradation, conventional thermal inkjet printers or low-heat inkjet printers can be used for practice of the present invention.

By using the above-described heatless printing process, the present invention successfully achieves delivery of multiple skin benefit agents with little or no reduction in their biological activities. First, the heatless printing process causes little or no degradation of the skin benefit agents. Second, certain skin benefit agents that are known to interfere with each other's biological activities can be placed into separate cartridges and deposited onto the substrate as separate droplets. More importantly, the droplets of such interfering skin benefit agents are sufficiently small in size that they can be arranged in a scattered manner. Consequently, such skin benefit agents can provide simultaneous treatments to the same region, but without having to be mixed with each other.

The cosmetic delivery sheet substrates so printed could be used anywhere on the face or body skin to predetermined areas for delivery of ingredients via a sheet material mask or patch or similar system. The exact size and shape of the cosmetic sheet will depend upon the intended use and product characteristics. The cosmetic sheets will have sufficient flexibility, and a size and shape adapted to conform to the desired treatment area of the user's skin. In a particularly preferred, but not necessary, embodiment of the present invention, the cosmetic sheet is a facial mask adapted to conform to facial features. It will be understood that a variety of shapes and sizes may be accommodated according to the invention. Such a cosmetic sheet may include a flexible substrate that is formed of, preferably but not necessarily, water-soluble materials, such as sugar or polysaccharides, collagen, and water-soluble film-forming polymers. The substrate contains multiple isolate, discrete regions, while at least two of such regions are imprinted with different skin benefit agents for treating different skin conditions according to the skin profile of the user.

As noted above, suitable skin benefit agents can be used in the present invention include, but are not limited to: anti-wrinkle or skin-tightening agents; anti-aging agents; moisturizing agents; skin-whitening or depigmentation agents; anti-inflammatory agents; anti-acne agents; DNA repair agents; skin lipid barrier repair agents; anti-cellulite agents; wound-healing agents; stretch-mark/scar removing agents; plumping agents; hair growth retardation agents; hair growth stimulating agents; dark cycle reduction or de-puffing agents; collagen synthesis or blood circulation enhancing agents; antioxidants; sebum-controlling agents; and pore-minimizing agents. Exemplary anti-wrinkle agents include, but are not limited to, acetyl hexapeptide-8, palmitoyl oligopeptide, dipeptide diaminobutyroyl, benzylamide diacetate, and the like. Exemplary skin-tightening agents include, but are not limited to, algae extract, pullulan, sweet almond seed extract, carbomer, palmitoyl oligopeptide, palmitoyl tetrapeptide-7, Quercus suber extract, and the like. Exemplary anti-aging agents include, but are not limited to, teprenone, trisodium resveratrol triphosphate, Polygonum cuspidatum root extract, whey protein, and the like. Exemplary moisturizing agents include, but are not limited to, hyaluronic acid, glycerin, urea, trehalose, and the like. Exemplary skin-whitening or depigmentation agents include, but are not limited to, ascorbic acid, magnesium ascorbyl phosphate, aminopropyl ascorbyl phosphate, mulberry root extract, Scutellaria baicalensis extract, grape extract, ferulic acid, hinokitol, and the like. Exemplary anti-inflammatory agents include, but are not limited to, spike moss extract, seal whip extract, Polygonum cuspidatum root extract, and the like. Exemplary anti-acne agents include, but are not limited to, salicylic acid, glycolic acid, lactobionic acid, and the like. Exemplary DNA repair agents include, but are not limited to, C1-C8 alkyl tetrahydroxycyclohexanoate, micrococcus lysate, bifida ferment lysate, and the like. Exemplary skin lipid barrier repair agents include, but are not limited to, phytosphingosine, linoleic acid, cholesterol, and the like. Exemplary anti-cellulite agents include, but are not limited to, Coleus forskohlii root extract, Magnolia grandiflora bark extract, Nelubo nucifera leaf extract, and the like. Exemplary wound-healing agents include, but are not limited to, Mimosa tenuiflora bark extract, soybean protein, and the like. Exemplary plumping agents include, but are not limited to, Saccharomyces/xylinum black tea ferment, Anemarrhena asphodeloides root extract, sodium hyaluronate, and the like. Exemplary hair growth retardation agents include, but are not limited to, ursolic acid, phytosphingosine, Boswella serrata extract, and the like. Exemplary hair growth stimulating agents include, but are not limited to, Serenoa serrulata fruit extract, licorice extract, acetyl glucosamine, and the like. Exemplary dark circle reduction or de-puffying agents include, but are not limited to, hesperidin methyl chalcone, dipeptide-2, Passiflora incarnate flower extract, linoleic acid, isolinoleic acid, and the like. Exemplary collagen synthesis or blood circulation enhancing agents include, but are not limited to, arginine, Ascophyllum nodosum extract, Asparagopsis armata extract, caffeine, and the like. Exemplary anti-oxidants include, but are not limited to, nordihydroguaiaretic acid, grape seed extract, green tea leaf extract, and the like.

The skin benefit agents as described hereinabove can be formulated into an ink formulation that is compatible with the printing device of the present invention. Such ink formulation may be an aqueous solution or an oil-in-water emulsion. When all the skin benefit agents to be delivered are water-soluble, it is preferred that the ink formulation is aqueous. When some of the skin benefit agents are oil-soluble, the ink formulation is preferably a micronized emulsion containing an oil phase in form of micronized oil droplets dispersed in a continuous aqueous phase.

Based on the particular skin conditions of the user, T-zone regions are imprinted with at least one sebum controlling agent for reducing the oiliness; at least one wrinkle reduction or skin-tightening agent for reducing fine lines and wrinkles is imprinted crow's foot region and frown line region at the corners of the user's eyes and mouth, respectively; under eye regions are imprinted with at least one dark circle reduction or de-puffying agent; and the chin region is imprinted with at least one anti-cellulite agent. Of course, the discrete regions themselves, may also be customized based on a skin profiling analysis. Customizations include size, shape and number of discrete regions. Preferably, but not necessarily, different regions on the mask may be marked with different colors for easy alignment with different facial features for which the skin benefit agents are intended.

As shown in FIGS. 9 and 11, multiple skin benefit agents may be printed in one region. These skin benefit agents are deposited onto the substrate as separate droplets, which are scattered among one another but without being mixed with one another. In this manner, such skin benefit agents can provide simultaneous treatment to the corners of the user's eyes and mouth with minimum or no interference with one another.

It will be understood by those skilled in the art that, while gel cosmetic sheets suitable for use in the present invention, are naturally tacky, a cosmetic sheet comprised of paper or a textile may require the presence of a cosmetically acceptable adhesive layer associated with the first surface of the support to enhance adherence to the skin. The adhesion of the sheet to the skin may occur via an adhesive compound associated with the surface of the sheet or it may be provided in the form of a gel or liquid, such as water, which moistens the sheet which then clings to skin. The user may also apply the mask to pre-moistened skin. It also is contemplated that a consumer could introduce a liquid activator to the sheet or to specific areas of the sheet which could serve to aid in adhesion of the sheet to the skin, to activate the impregnated formulation, or both. The cosmetic treatment delivery sheet may also be provided with a supporting third substrate sheet which can be removed, e.g. peeled away, before the sheet is applied to the skin.

The cosmetic treatment delivery sheet, including the first sheet substrate 2 and/or the second sheet substrate 44, may be formed of any thin, porous, flexible absorbent material, including woven and non-woven fabrics, including felts, paper, natural fibers, synthetic fibers, elastic blends or a mixture thereof. Non-limiting examples include cotton, linen, rayon, thermoplastics, and cellulosics. The sheet material may be a water-soluble material, such as sugar or polysaccharides, collagen, and water-soluble film-forming polymers. The sheet material may also comprise a gel, such as a hydrogel, comprised of, for example, agarose or water-soluble low-substituted cellulose ether which may include methyl cellulose, hydroxyethyl cellulose, hydroxypropyl cellulose, hydroxypropylmethyl cellulose, hydroxypropylhydroxyethyl cellulose, hydroxyethylmethyl cellulose, ethyl cellulose, hydroxyethylethyl cellulose, or carboxymethyl cellulose. Non-woven fabrics are especially preferred from the viewpoints of cost, productivity and aesthetic feel. Examples of preferred non-woven materials include, but are not limited to, natural and synthetic felts, rice paper or cloth, and bamboo cloth. In a preferred but not necessary embodiment of the present invention, both the substrates and the skin benefit agents of the cosmetic sheet are completely water-soluble, such as sugar or collagen, so upon application of water or like liquid activator, the cosmetic sheet softens and conforms to the skin, and subsequently, the entire sheet is absorbed by the skin surface without having to be removed. Commercially available cosmetic sheets suitable for practice of the present invention include, for example, the sugar-based Frosting Sheets from Kopykake (Torrance, Calif.), the Matricol® Collagen Sheets from Dr. Suwelack Skin & Health Care AG (Billerbeck, Germany), and the 3M™ Transparent 2.6 mil Polyethylene Medical Tape 9830.

### Example 1

### Targeted Delivery of Caffeine Power

An aqueous solution containing caffeine power was prepared by mixing the following ingredients together:

| Ingredients | wt % |
|---|---|
| Deionized water | QS |
| Butylene glycol | 15.00 |
| Caffeine | 5.00 |
| FD&C Blue No. 1 | 0.04 |
| Phenoxyethanol | 0.50 |
| Total | 100.00 |

The FD&D Blue No. 1 color was provided to mark regions with caffeine power printed thereon. The aqueous solution as described hereinabove was placed into a refillable ink cartridge of an Epson Workforce 30 inkjet printer, which was in turn connected to a personal computer installed with Photoshop Element 4.0. Three different types of first sheet substrate sheets, including a sugar-based Frosting Sheet from Kopykake (Torrance, Calif.), a Matricol® Collagen Sheet from Dr. Suwelack Skin & Health Care AG (Billerbeck, Germany), and a 3M™ Transparent 2.6 mil Polyethylene Medical Tape 9830 were fed to the paper tray of the Epson Workforce 30 inkjet printer, and the caffeine-containing aqueous solution was successfully printed onto the substrate sheets by the inkjet printer. The first sheet substrate sheets were provided with a micro-current generator printed directly on the first substrate sheet, or printed separately on a second substrate sheet that was subsequently attached to the first substrate sheet. The resulting cosmetic treatment delivery sheets were then activated to generate micro-current and tested for successful targeted delivery of caffeine as a skin benefit agent.

### Example 2

### Targeted Delivery of Salicylic Acid

An aqueous solution containing salicylic acid was prepared by mixing the following ingredients together:

| Ingredients | wt % |
|---|---|
| Deionized water | QS |
| Isopentyldiol | 40.00 |
| Salicylic acid | 1.00 |
| FD&C Yellow No. 5 | 0.04 |
| Phenoxyethanol | 0.50 |
| Total | 100.00 |

The FD&D Yellow No. 5 color was provided to mark regions with salicylic acid (SA) printed thereon. The aqueous solution as described hereinabove was placed into a refillable ink cartridge of an Epson Workforce 30 inkjet printer, which was in turn connected to a personal computer installed with Photoshop Element 4.0. Three different types of substrate sheets, including a sugar-based Frosting Sheet from Kopykake (Torrance, Calif.), a Matricol® Collagen Sheet from Dr. Suwelack Skin & Health Care AG (Billerbeck, Germany), and a 3M™ Transparent 2.6 mil Polyethylene Medical Tape 9830 were fed to the paper tray of the Epson Workforce 30 inkjet printer, and the SA-containing aqueous solution was successfully printed onto the substrate sheets by the inkjet printer. The first sheet substrate sheets were provided with a micro-current generator printed directly on the first substrate sheet, or printed separately on a second substrate sheet that was subsequently attached to the first substrate sheet. The resulting cosmetic treatment delivery sheets were then activated to generate micro-current and tested for successful targeted delivery of SA as a skin benefit agent.

Although the present invention has been described in accordance with preferred embodiments, it will be understood by those skilled in the art that various changes and modifications may be made without departing from the spirit and scope of the invention.

1. A cosmetic treatment delivery system for targeted and individualized skin treatment of at least a first skin condition and a second skin condition in a predetermined treatment area of a user's skin, the system comprising:
a first sheet substrate arranged and constructed for conforming to the predetermined treatment area of the user's skin, the substrate having defined thereon at least a first isolate, discrete region imprinted with a first skin benefit agent for treating the first skin condition, the first isolate, discrete region imprinted on the first sheet substrate in a first position corresponding to a first respective location of the first skin condition in the predetermined treatment area of the user's skin, and a second isolate, discrete region imprinted with a second skin benefit agent for treating the second skin condition, the second isolate, discrete region imprinted on the first sheet substrate in a second position corresponding to a second respective location of the second skin condition in the predetermined treatment area of the user's skin, the first sheet substrate further having one of an oxidation agent or reduction agent imprinted thereon; and
an activating cream containing the other of the oxidation agent or reduction agent, the activating cream adapted to be applied to or with the first sheet substrate to cause a microcurrent to be generated in a quantity sufficient to facilitate delivery of at least one of the first skin benefit agent and the second skin benefit agent, the microcurrent caused to be delivered through at least one of the first isolate, discrete region or the second isolate, discrete region of the first sheet substrate.
